(19) United States
(12) Patent Application Publication
(10) Pub. No.: US 2019/0256477 A1
(43) Pub. Date: Aug. 22, 2019

(54) METHOD FOR THE PREPARATION OF 3-(TRIFLUOROMETHYL)PYRAZINE-2-CARBOXYLIC ACID ESTERS

(71) Applicant: LONZA LTD, Visp (CH)

(72) Inventors: Florencio ZARAGOZA DOERWALD, Visp (CH); Christoph TAESCHLER, Visp (CH); Erick CARREIRA, Zuerich (CH)

(21) Appl. No.: 16/320,243

(22) PCT Filed: Aug. 30, 2017
(86) PCT No.: PCT/EP2017/071690

§ 371 Date: Jan. 24, 2019

Related U.S. Application Data
(60) Provisional application No. 62/382,446, filed on Sep. 1, 2016.

(30) Foreign Application Priority Data
Sep. 1, 2016 (EP) 16186871.6
Dec. 13, 2016 (EP) 16203697.4
Feb. 21, 2017 (EP) 17157057.5

(51) Int. Cl. C07D 241/24

(52) U.S. Cl. CPC C07D 241/24

(57) ABSTRACT

The invention discloses a method for the preparation of 3-(trifluoromethyl)pyrazine-2-carboxylic acid esters starting from alkyl 4,4,4-trifluoro-2-(hydroxyimino)-3-oxobutanoates by reaction with ethylenediamine.

The invention discloses a method for the preparation of 3-(trifluoromethyl)pyrazine-2-carboxylic acid esters starting from alkyl 4,4,4-trifluoro-2-(hydroxyimino)-3-oxobutanoates by reaction with ethylenediamine.

### BACKGROUND OF THE INVENTION

3-(Trifluoromethyl)pyrazine-2-carboxylic acid esters as well as the corresponding acid are important intermediates for the preparation of biologically active compounds, such as drugs or agrochemicals.

One specific example of an important, biologically active compound requiring such esters as synthetic intermediates is the fungicide pyraziflumid (CAS 942515-63-1).

A number of different preparation methods of such esters have been disclosed, but all of them require more than three synthetic steps, are based on expensive starting materials, or require the use of expensive or dangerous reagents. For instance, WO 2010/122794 A1 discloses the conversion of 4,4,4-trifluoro-3-oxo-2-halobutanoates into such esters by treatment with sodium azide, followed by reaction with ethylenediamine and dehydrogenation with a noble metal catalyst. Sodium azide is a hazardous reagent, and requires costly safety precautions when handled on a large scale. Noble metal catalysts are expensive, and traces of noble metals are sometimes difficult to remove from the products of noble-metal-catalyzed reactions.

U.S. Pat. No. 5,374,615 A1 discloses the nitrosation of 4,4,4-trifluoro-3-oxobutanoates with sodium nitrite in acetic acid, as specific example the preparation of 4,4,4-trifluoro-2-(hydroxyimino)-3-oxobutanoic acid, ethyl ester, is described in example 21 A.

There was a need for a method for preparation of such esters that does not require more than three synthetic steps, that is not based on expensive starting materials, and that does not require the use of expensive or dangerous, such as toxic or explosive reagents, such as noble metal catalysts or sodium azide.

A preparation method for such esters was found that is short, that is based on readily available starting materials, that does not require toxic or explosive reagents or intermediates, and that is well suited for the large scale preparation of such esters. The method can be done as a one-pot method without mandatorily requiring any isolation of an intermediates, the method can be done without change of a solvent.

### SUMMARY OF THE INVENTION

Subject of the invention is a method for the preparation of compound of formula (I);

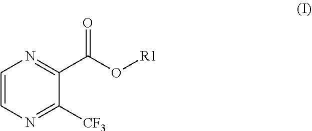

the method comprises two steps, a step ST1 and a step ST2;

ST1 comprises a reaction REAC1, wherein compound of formula (II)

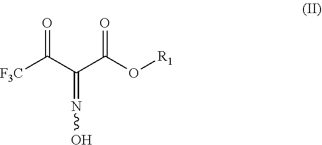

is reacted with ethylenediamine in the presence of an acid ACID1 and of a reducing agent REDUC1;
ST2 comprises a reaction REAC2, wherein the reaction product of REAC1 is reacted with an oxidant OXI2;
R1 is C₁₋₄ alkyl;
ACID1 is selected from the group consisting of BF₃OEt₂, hydroxybenzotriazole, nicotinic acid, nicotinic acid N-oxide, N-hydroxysuccinimide, formic acid, C₁₋₆ alkanoic acid, benzoic acid, benzoic acid substituted by Cl or OH, dichloroacetic acid, trifluoroacetic acid, trichloroacetic acid, toluenesulfonic acid, methanesulfonic acid, hydrochloric acid, sulfuric acid, and phosphoric acid;
REDUC1 is selected from the group consisting of zinc, iron, aluminum, magnesium, tin, SnCl₂, NaBH₄, hydrogen, PCl₃, H₃PO₂, P(R10)(R11)(R12) and P(OR20)(OR21)(OR22);
R10, R11, and R12 are identical or different and independently from each other selected from the group consisting of phenyl, tolyl, cyclohexyl, butyl, adamantyl, 2-carboxyethyl, and methyl;
R20, R21 and R22 are identical or different and independently from each other selected from the group consisting of H, C₁₋₈ alkyl, C₃₋₈ cycloalkyl, or phenyl;
OXI2 is selected from the group consisting of chlorine, bromine, iodine, N-chlorosuccinimide, N-bromosuccinimide, N-chlorophthalimide, N-bromophthalimide, N,N′,N″-trichloroisocyanuric acid, 1,3-dichloro-5,5-dimethylhydantoin, 1,3-dibromo-5,5-dimethylhydantoin, sodium hypochlorite, calcium hypochlorite, nitric acid, nitrous acid, sulfur, R30-OOH, air, and oxygen;

R30 is H or C₁₋₆ alkyl, the C₁₋₆ alkyl is unsubstituted or substituted with phenyl.

### DETAILED DESCRIPTION OF THE INVENTION

BF₃OEt₂ is boron trifluoride diethyletherate.
Compound of formula (II) can be used both as E and as Z isomer, it can be used as in hydrate form or in anhydrous form, or it can be used as a mixture of any of these forms and isomers.
Preferably, R1 is methyl or ethyl;
more preferably, R1 is ethyl.
Preferably, the molar amount of ethylenediamine is from 5 to 0.5 times, more preferably from 3 to 1 time, even more preferably from 2 to 1 time, of the molar amount of compound of formula (II).
Preferably, ACID1 is selected from the group consisting of BF₃OEt₂, hydroxybenzotriazole, nicotinic acid, nicotinic acid N-oxide, N-hydroxysuccinimide, formic acid, acetic acid, propionic acid, n-butyric acid, iso-butyric acid, 2-methylpentanoic acid, pivalic acid, benzoic acid, chlorobenzoic acid, 2-hydroxybenzoic acid, dichloroacetic acid, trifluoroacetic acid, toluenesulfonic acid, methanesulfonic acid, and hydrochloric acid;
more preferably, ACID1 is selected from the group consisting of BF₃OEt₂, hydroxybenzotriazole, N-hydroxysuccinimide, formic acid, acetic acid, propionic acid, iso-butyric acid, pivalic acid, benzoic acid, chlorobenzoic acid, dichloroacetic acid, trifluoroacetic acid, toluenesulfonic acid, methanesulfonic acid, and hydrochloric acid;
even more preferably, ACID1 is selected from the group consisting of formic acid, acetic acid, propionic acid, iso-butyric acid, pivalic acid, benzoic acid, dichloroacetic acid, trifluoroacetic acid, toluenesulfonic acid, methanesulfonic acid, and hydrochloric acid;
especially, ACID1 is selected from the group consisting of acetic acid, propionic acid, iso-butyric acid, benzoic acid, dichloroacetic acid, trifluoroacetic acid, toluenesulfonic acid, methanesulfonic acid, and hydrochloric acid.

In another preferred embodiment,
ACID1 is selected from the group consisting of formic acid, acetic acid, propionic acid, butyric acid, pivalic acid, benzoic acid, trifluoroacetic acid, trichloroacetic acid, toluenesulfonic acid, methanesulfonic acid, hydrochloric acid, sulfuric acid, and phosphoric acid;
more preferably, ACID1 is selected from the group consisting of formic acid, acetic acid, propionic acid, trifluoroacetic acid, trichloroacetic acid, methanesulfonic acid, hydrochloric acid, and sulfuric acid;
even more preferably, ACID1 is selected from the group consisting of acetic acid, propionic acid, trifluoroacetic acid, hydrochloric acid, and sulfuric acid;
especially, ACID1 is selected from the group consisting of acetic acid, propionic acid, and sulfuric acid.
Preferably, the molar amount of ACID1 is from 20 to 0.3 times, more preferably from 10 to 0.5 times, even more preferably from 5 to 1 time, of the molar amount of compound of formula (II).
Preferably, REDUC1 is selected from the group consisting of zinc, aluminum, magnesium, hydrogen, PCl₃, H₃PO₂, P(R10)(R11)(R12) and P(OR20)(OR21)(OR22);
more preferably, REDUC1 is selected from the group consisting of zinc, hydrogen, PCl₃, H₃PO₂, P(R10)(R11)(R12) and P(OR20)(OR21)(OR22).
In another preferred embodiment, REDUC1 is selected from the group consisting of zinc, iron, aluminum, magnesium, tin, SnCl₂, NaBH₄, hydrogen, P(R10)(R11)(R12) and P(OR20)(OR21)(OR22);
more preferably, REDUC1 is selected from the group consisting of zinc, aluminum, magnesium, hydrogen, P(R10)(R11)(R12)and P(OR20)(OR21)(OR22);
even more preferably, REDUC1 is selected from the group consisting of zinc, hydrogen, P(R10)(R11)(R12) and P(OR20)(OR21)(OR22).
Preferably, R10, R11, and R12 are identical or different and independently from each other selected from the group consisting of phenyl, tolyl, cyclohexyl, butyl, adamantyl, and methyl;
more preferably, R10, R11, and R12 are identical or different and independently from each other selected from the group consisting of phenyl and butyl.
Preferably, R20, R21 and R22 are identical or different and independently from each other selected from the group consisting of H, C₁₋₈ alkyl and phenyl;
more preferably, R20, R21 and R22 are identical or different and independently from each other selected from the group consisting of H, C₁₋₄ alkyl and phenyl.
In another preferred embodiment, R20, R21 and R22 are identical or different and independently from each other selected from the group consisting of C₁₋₈ alkyl, C₃₋₈ cycloalkyl, or phenyl;
more preferably, R20, R21 and R22 are identical or different and independently from each other selected from the group consisting of C₁₋₈ alkyl and phenyl;
more preferably, R20, R21 and R22 are identical or different and independently from each other selected from the group consisting of C₁₋₄ alkyl and phenyl.
Particular embodiments of REDUC1 are selected from the group consisting of trimethyl phosphite and triethyl phosphite.
In case that REDUC1 is hydrogen, then REAC1 can be done in the presence of a catalyst CAT1, CAT1 is selected from the group consisting of palladium, platinum, rhodium, nickel, and ruthenium;
preferably, CAT1 is selected from the group consisting of palladium, platinum, rhodium, and ruthenium;
more preferably, CAT1 is selected from the group consisting of palladium, platinum, or ruthenium.
CAT1 can be unsupported or supported on a suitable support such as charcoal or alumina.
Preferably, the molar amount of REDUC1 is from 10 to 0.5 times, more preferably from 5 to 1 time, even more preferably from 3 to 1 times, of the molar amount of compound of formula (II).
More preferably, and this in particular for embodiments where REDUC1 is trimethyl phosphite or triethyl phosphite, the molar amount of REDUC1 is from 2.75 to 1.25 times, more preferably from 2.5 to 1.5 times, and even more preferably from 2.2 to 1.8 times, of the molar amount of compound of formula (II).
REAC1 can be done in a solvent SOLV1, SOLV1 is preferably selected from the group consisting of acetonitrile, valeronitrile, dioxane, tert-butyl methyl ether, toluene, chlorobenzene, sulfolan, N,N-dimethylformamide, N,N-dimethylacetamide, 2-methyltetrahydrofuran, tetrahydrofuran, ethyl acetate, isopropyl acetate, butyl acetate, pyridine, 2-methylpyridine, 3-methylpyridine, 4-methylpyridine, 2-methyl-5-ethylpyridine, 2,4,6-trimethylpyridine, dichloromethane, chloroform, carbontetrachloride, and mixtures thereof;
more preferably, SOLV1 is selected from the group consisting of acetonitrile, valeronitrile, dioxane, toluene, 2-methyltetrahydrofuran, ethyl acetate, isopropyl acetate, butyl acetate, pyridine, 2-methylpyridine, 3-methylpyridine, 4-methylpyridine, 2-methyl-5-ethylpyridine, 2,4,6-trimethylpyridine, dichloromethane, chloroform, carbontetrachloride, and mixtures thereof;
even more preferably, SOLV1 is selected from the group consisting of acetonitrile, dioxane, toluene, 2-methyltetrahydrofuran, butyl acetate, pyridine, 2-methylpyridine, 3-methylpyridine, 4-methylpyridine, 2-methyl-5-ethylpyridine, dichloromethane, chloroform, carbontetrachloride, and mixtures thereof;
especially, SOLV1 is selected from the group consisting of acetonitrile, dioxane, toluene, 2-methyltetrahydrofuran, butyl acetate, pyridine, 3-methylpyridine, dichloromethane, chloroform, carbontetrachloride, and mixtures thereof.
Preferably, REAC1 is done in SOLV1.
Preferably, the reaction temperature TEMP1 of REAC1 is from −80 to 200° C., more preferably from −30 to 150° C., even more preferably from −20 to 100° C.
Preferably, the pressure PRESS1 of REAC1 is adjusted according to the vapor pressure of the reaction mixture at the chosen TEMP1 of REAC1; but PRESS1 can also be adjusted to a higher pressure than the vapor pressure of the reaction mixture at the chosen TEMP1. A PRESS1 higher than the vapor pressure of the reaction mixture at the chosen TEMP1 can be adjusted for example by applying inert gas such a nitrogen or argon to the reaction vessel;
more preferably, REAC1 is done at a pressure from atmospheric pressure to 20 bar, more preferably from atmospheric pressure to 10 bar, even more preferably from atmospheric pressure to 5 bar.
Preferably, the reaction time TIME1 of REAC1 is from 30 min to 48 h, more preferably from 1 h to 24 h, and even more preferably from 1 h to 12 h.
REAC1 can be performed under an atmosphere of air or under an atmosphere of an inert gas, such as nitrogen or such as a noble gas, such as argon.
Preferably, OXI2 is selected from the group consisting of chlorine, bromine, iodine, N-bromosuccinimide, N,N′,N″-trichloroisocyanuric acid, 1,3-dichloro-5,5-dimethylhydantoin, 1,3-dibromo-5,5-dimethylhydantoin, sodium hypochlorite, calcium hypochlorite, nitric acid, nitrous acid, R30-OOH, air, and oxygen;
more preferably, OXI2 is selected from the group consisting of chlorine, bromine, iodine, N-bromosuccinimide, N,N′,N″-trichloroisocyanuric acid, 1,3-dichloro-5,5-dimethylhydantoin, and 1,3-dibromo-5,5-dimethylhydantoin, sodium hypochlorite, R30-OOH, air, and oxygen.
In another preferred embodiment, OXI2 is selected from the group consisting of chlorine, bromine, iodine, N-chlorosuccinimide, N-bromosuccinimide, N-chlorophthalimide, N-bromophthalimide, N,N′,N″-trichloroisocyanuric acid, 1,3-dichloro-5,5-dimethylhydantoin, 1,3-dibromo-5,5-dimethylhydantoin, sodium hypochlorite, calcium hypochlorite, nitric acid, nitrous acid, sulfur, hydrogen peroxide, air, and oxygen;
more preferably, OXI2 is selected from the group consisting of chlorine, bromine, iodine, N-bromosuccinimide, N,N′,N″-trichloroisocyanuric acid, 1,3-dichloro-5,5-dimethylhydantoin, 1,3-dibromo-5,5-dimethylhydantoin, sodium hypochlorite, calcium hypochlorite, nitric acid, nitrous acid, hydrogen peroxide, air, and oxygen;
more preferably, OXI2 is selected from the group consisting of chlorine, bromine, iodine, N-bromosuccinimide, N,N′,N″-trichloroisocyanuric acid, 1,3-dichloro-5,5-dimethylhydantoin, and 1,3-dibromo-5,5-dimethylhydantoin, sodium hypochlorite;
even more preferably, OXI2 is selected from the group consisting of chlorine, bromine, N-bromosuccinimide, N,N′,N″-trichloroisocyanuric acid, 1,3-dichloro-5,5-dimethylhydantoin, and 1,3-dibromo-5,5-dimethylhydantoin, sodium hypochlorite.
In case that OXI2 is oxygen, then REAC2 can be done in the presence of a catalyst CAT2, CAT2 can for example be selected from the group consisting of platinum, bismuth, nickel, molybdenum, and ruthenium;
preferably, CAT2 can for example be selected from the group consisting of platinum, molybdenum, and ruthenium.
CAT2 can be unsupported or supported on a suitable support such as charcoal or alumina.
Preferably, the molar amount of OXI2 is from 2 to 20 times, more preferably from 2 to 10 time, even more preferably from 2 to 7.5 times, of the molar amount of compound of formula (II).

Preferably, R30 is H or C₃₋₆ alkyl, the C₃₋₆ alkyl is unsubstituted or substituted with phenyl; more preferably, R30 is H, C₄₋₆ alkyl hydroperoxide or cumene hydroperoxide; even more preferably, R30 is is H, C₄ alkyl hydroperoxide or cumene hydroperoxide. especially, R30 is is H.
REAC2 can be done in the presence of a catalyst OXICAT2, OXICAT2 is any known catalyst that is used to catalyze an oxidation reaction with OXI2 and oxidant;
preferably, OXICAT2 is a system derived from I₂/I⁻ or WO₄²⁻.
More preferably the system derived from I₂/I²⁻ is used in form of a tetraalkylammonium iodide, the alkyl being preferably a C₁₋₁₂ alkyl, such as tetrabutylammonium iodide.
More preferably the system derived from WO₄²⁻ is used in form of Na₂WO₄.
REAC2 can be done in a solvent SOLV2, SOLV2 is preferably selected from the group consisting of acetonitrile, valeronitrile, dioxane, tert-butyl methyl ether, toluene, chlorobenzene, sulfolan, N,N-dimethylformamide, N,N-dimethylacetamide, 2-methyltetrahydrofuran, tetrahydrofuran, ethyl acetate, isopropyl acetate, butyl acetate, pyridine, 2-methylpyridine, 3-methylpyridine, 4-methylpyridine, 2-methyl-5-ethylpyridine, 2,4,6-trimethylpyridine, dichloromethane, chloroform, carbontetrachloride, and mixtures thereof;
more preferably, SOLV2 is selected from the group consisting of acetonitrile, valeronitrile, dioxane, toluene, 2-methyltetrahydrofuran, ethyl acetate, isopropyl acetate, butyl acetate, pyridine, 2-methylpyridine, 3-methylpyridine, 4-methylpyridine, 2-methyl-5-ethylpyridine, 2,4,6-trimethylpyridine, dichloromethane, chloroform, carbontetrachloride, and mixtures thereof;
even more preferably, SOLV2 is selected from the group consisting of acetonitrile, dioxane, toluene, 2-methyltetrahydrofuran, pyridine, 2-methylpyridine, 3-methylpyridine, 4-methylpyridine, 2-methyl-5-ethylpyridine, dichloromethane, chloroform, carbontetrachloride, and mixtures thereof;
especially, SOLV2 is selected from the group consisting of acetonitrile, dioxane, toluene, 2-methyltetrahydrofuran, pyridine, 3-methylpyridine, dichloromethane, chloroform, carbontetrachloride, and mixtures thereof.
Preferably, REAC2 is done in SOLV2.
Preferably, the reaction temperature TEMP2 of REAC2 is from −80 to 200° C., more preferably from −30 to 150° C., even more preferably from −20 to 100° C.
Preferably, the pressure PRESS2 of REAC2 is adjusted according to the vapor pressure of the reaction mixture at the chosen TEMP2 of REAC2; but PRESS2 can also be adjusted to a higher pressure than the vapor pressure of the reaction mixture at the chosen TEMP2. A PRESS2 higher than the vapor pressure of the reaction mixture at the chosen TEMP2 can be adjusted for example by applying inert gas such a nitrogen or argon to the reaction vessel;
more preferably, REAC2 is done at a pressure from atmospheric pressure to 20 bar, more preferably from atmospheric pressure to 10 bar, even more preferably from atmospheric pressure to 5 bar.
Preferably, the reaction time TIME2 of REAC2 is from 10 min to 48 h, more preferably from 30 min to 24 h.
REAC1 can be performed under an atmosphere of air or under an atmosphere of an inert gas, such as nitrogen or such as a noble gas, such as argon.
After REAC2, compound of formula (I) can be isolated and purified by conventional methods, which are known to those skilled in the art. These conventional methods include extraction, distillation, preferably fractional distillation, which can be done under reduced pressure, crystallization, chromatography, or any combination of these methods of purification.
Preferably, REAC1 and REAC2 are done in the same solvent.
Preferably, REAC1 and REAC2 are done consecutively in the same reaction vessel.
Preferably, the reaction product of REAC1 is not isolated. Preferably, REAC1 and REAC2 are done in form of one-pot-reaction.
Compounds of formula (II) are available by known methods, such as for example by nitrosation of the respective oxobutanoates with sodium nitrite in acetic acid.
Preferably, compound of formula (II) is prepared in a step ST0;
ST0 comprises a reaction REAC0, wherein compound of formula (III)

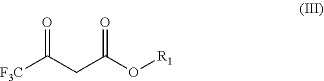

is reacted with NITR0 in the presence of ACID0;
with R1 as defined herein, also with all its embodiments;
NITR0 is selected from the group consisting of NaNO₂, ClNO, nitrosylsulfuric acid and R40-O—NO;
R40 is C₁₋₁₀ alkyl;
ACID0 is selected from the group consisting of ACID1 and chlorides and anhydrides of C₂₋₄ alkanoic acid.
Preferably, NITR0 is selected from the group consisting of NaNO₂, nitrosylsulfuric acid and R40-O—NO; and
R40 is C₁₋₅ alkyl.
An embodiment of NITR0 is butyl nitrite, preferably n-butyl nitrite.
More preferably, NITR0 is NaNO₂ or n-butyl nitrite.
Preferably, the molar amount of NITR0 is from 1 to 5 times, more preferably from 1 to 3 times, even more preferably from 1 to 1.5 time, of the molar amount of compound of formula (III).
NITR0 can be used in form of an aqueous solution;
preferably it is used in anhydrous form
Preferably, ACID0 is selected from the group consisting of ACID1 and chloride and anhydride of acetic acid;
more preferably, ACID0 is chloride or anhydride of acetic acid or ACID1 with ACID1 in its various embodiments as defined herein; preferably ACID1 is acetic acid, trifluoroacetic acid, or hydrochlorid acid, more preferably ACID1 is acetic acid or hydrochlorid acid.
Preferably, the molar amount of ACID0 is from 0.01 to 10 times, more preferably from 0.1 to 5 times, even more preferably from 0.2 to 5 time, of the molar amount of NITR0.
Preferably, the reaction temperature TEMP0 of REAC0 is from −20 to 20° C., more preferably from −10 to 20° C.
Preferably, the pressure PRESS0 of REAC0 is adjusted according to the vapor pressure of the reaction mixture at the chosen TEMP0 of REAC0; but PRESS0 can also be adjusted to a higher pressure than the vapor pressure of the reaction mixture at the chosen TEMP0. A PRESS0 higher than the vapor pressure of the reaction mixture at the chosen TEMP0 can be adjusted for example by applying inert gas such a nitrogen or argon to the reaction vessel;
more preferably, REAC0 is done at a pressure from atmospheric pressure to 20 bar, more preferably from atmospheric pressure to 10 bar.
Preferably, the reaction time TIME0 of REAC0 is from 10 min to 12 h, more preferably from 10 min to 8 h.
REAC0 can be done in a solvent SOLV0, SOLV0 is preferably selected from the group consisting of acetonitrile, valeronitrile, dioxane, tert-butyl methyl ether, toluene, chlorobenzene, sulfolan, N,N-dimethylformamide, N,N-dimethylacetamide, 2-methyltetrahydrofuran, tetrahydrofuran, ethyl acetate, isopropyl acetate, butyl acetate, dichloromethane, chloroform, carbontetrachloride, and mixtures thereof;
more preferably, SOLV0 is selected from the group consisting of acetonitrile, valeronitrile, dioxane, toluene, 2-methyltetrahydrofuran, ethyl acetate, isopropyl acetate, butyl acetate, dichloromethane, chloroform, carbontetrachloride, and mixtures thereof;
even more preferably, SOLV0 is selected from the group consisting of acetonitrile, dioxane, toluene, 2-methyltetrahydrofuran, dichloromethane, chloroform, and mixtures thereof;
especially, SOLV0 is selected from the group consisting of acetonitrile, dioxane, toluene, 2-methyltetrahydrofuran, dichloromethane, chloroform, and mixtures thereof.
SOLV0 can be exchanged for SOLV1 after REAC0, e.g. SOLV0 can be removed by distillation after REAC0.
After REAC0, compound of formula (II) can be isolated by standard methods. In case that NITR0 is aqueous NaNO₂, compound of formula (II) is obtained in form of a hydrated oxime and can be dehydrated for example by stirring a solution of the hydrated oxime in dichloromethane with the 2 to 10 fold amount by weight, based on the weight of compound of formula (II), of anhydrous calcium chloride for 12 to 36 h at room temperature, followed by filtration and evaporation of the dichloromethane.
REAC0, REAC1 and REAC2 can be done in the same solvent.
Preferably, REAC0, REAC1 and REAC2 are done consecutively in the same reaction vessel.
Preferably, the reaction product of REAC0 is not isolated. Preferably, REAC0, REAC1 and REAC2 are done in form of one-pot-reaction, preferably without isolation of compound of formula (II).
The one pot reaction without isolation of compound of formula (II) is a preferred embodiment in case that NITR0 is n-butyl nitrite.

Preferably, REAC0 is done in the absence of water.

### EXAMPLES

Internal standard for ¹H NMR: Triisobutyl phosphate, if not otherwise stated

### Example 1

### Ethyl 4,4,4-trifluoro-2-(hydroxyimino)-3-oxobutanoate

To a mixture of acetic acid (30 ml, 0.5 mol) and ethyl 4,4,4-trifluoro-3-oxobutanoate (9.21 g, 50.0 mmol) at 15° C. a solution of sodium nitrite (4.52 g, 65.5 mmol) in water (7.0 ml) was added dropwise within 10 min. After stirring at room temperature for 1.5 h the mixture was diluted with ethyl acetate (100 ml) and, while stirring and cooling to maintain the temperature below 30° C., a solution of sodium hydroxide (16 g, 0.4 mol) in water (200 ml) was added. Phases were separated, and NaHCO₃ (1 g) was added to the aqueous phase. The aqueous phase was then extracted with ethyl acetate (two times 50 ml), and the combined organic phases were dried (MgSO₄) and concentrated under reduced pressure. The product was obtained as an oil (9.60 g, 91% pure by ¹H NMR; yield: 82%), which slowly and partially solidified. According to the NMR spectra this oil consisted of an equimolar mixture of the hydrated and the non-hydrated form.
¹H NMR (DMSO, 400 MHz) delta=14.66 (s, br, 0.4H), 12.04 (s, 0.4H), 7.82 (s, 0.76H), 4.34 (quart, J=7 Hz, 1H), 4.20 (quart, J=7 Hz, 1H), 1.25 (m, 3H).
¹³C NMR (DMSO, 100 MHz) delta=174.9 (quart, J=36 Hz), 161.7, 159.5, 149.6, 146.7, 122.4 (quart, J=288 Hz), 115.6 (quart, J=288 Hz), 91.0 (quart, J=36 Hz), 62.1, 13.7.
¹⁹F NMR (DMSO, 376.5 MHz) delta=−71.5 (s), −82.0 (s).

### Example 2

### Ethyl 3-(trifluoromethyl)pyrazine-2-carboxylate with AcOH

To ethyl 4,4,4-trifluoro-2-(hydroxyimino)-3-oxobutanoate (0.11 g, approx 0.5 mmol, prepared according to Example 1) were added at room temperature in the following order acetic acid (0.09 ml, 1.5 mmol), pyridine (0.80 ml, 10 mmol), ethylenediamine (0.05 ml, 0.75 mmol), and triethyl phosphite (0.129 ml, 0.75 mmol). The mixture was stirred at room temperature for 6 h, cooled to 15° C., and bromine (0.128 ml, 2.5 mmol) was added dropwise while stirring. The mixture was stirred at room temperature for 16 h. Analysis of a sample by ¹⁹F NMR indicated that the title pyrazine had been formed in approximately 50% yield. The product ethyl 3-(trifluoromethyl)pyrazine-2-carboxylate was isolated by addition of aqueous 1N HCl (10 ml), saturation with NaCl, and extraction with ethyl acetate (3 ml). Addition of iBu₃PO₄ (0.05 ml), concentration of a sample of the ethyl acetate extract, and analysis by ¹H NMR indicated, that the title pyrazine ethyl 3-(trifluoromethyl)pyrazine-2-carboxylate had been formed in 44% yield.
¹H NMR (DMSO, 400 MHz) delta=9.08 (m, 2H), 4.46 (quart, J=7 Hz, 2H), 1.35 (t, J=7 Hz, 3H).
¹⁹F NMR (DMSO, 376.5 MHz) delta=−64.3.

### Example 3

### Oxidation with Bu₄NI/tBuOOH

To ethyl 4,4,4-trifluoro-2-(hydroxyimino)-3-oxobutanoate (0.176 g, approx 0.75 mmol, prepared according to Example 1) were added at 0° C. in the following order acetic acid (0.18 ml, 3.0 mmol), pyridine (1.2 ml, 15 mmol), ethylenediamine (0.065 ml, 0.98 mmol), and triethyl phosphite (0.193 ml, 1.13 mmol). The mixture was stirred at 0° C. for 1 h, and then at room temperature for 16 h. Tetrabutylammonium iodide (54 mg, 0.15 mmol) and tert-butylhydroperoxide (70 wt-% in water, 0.416 ml, 3.0 mmol) were then added, and stirring at room temperature was continued for 7 h. The mixture was then diluted with 1 N aqueous hydrochloric acid, saturated with sodium chloride, and extracted with ethyl acetate (3 ml). To the extract was added triisobutylphosphate (0.05 ml, 0.182 mmol) as internal standard. Analysis by 1H NMR indicated, that ethyl 3-(trifluoromethyl)pyrazine-2-carboxylate had been formed in 37% yield.

### Example 4

### Oxidation with Na₂WO₄/tBuOOH

To ethyl 4,4,4-trifluoro-2-(hydroxyimino)-3-oxobutanoate (0.176 g, approx 0.75 mmol, prepared according to Example 1) were added at 0° C. in the following order acetic acid (0.18 ml, 3.0 mmol), pyridine (1.2 ml, 15 mmol), ethylenediamine (0.065 ml, 0.98 mmol), and triethyl phosphite (0.193 ml, 1.13 mmol). The mixture was stirred at 0° C. for 1 h, and then at room temperature for 22 h. The mixture was then cooled to −30° C., and sodium tungstate hydrate (Na₂WO₄—H₂O, 50 mg, 0.15 mmol) and tert-butylhydroperoxide (70 wt-% in water, 0.416 ml, 3.0 mmol) were then added, and the mixture was stirred at room temperature for 9 h. The mixture was then diluted with 1 N aqueous hydrochloric acid, saturated with sodium chloride, and extracted with ethyl acetate (3 ml). To the extract was added triisobutylphosphate (0.05 ml, 0.182 mmol) as internal standard. Analysis by 1H NMR indicated, that ethyl 3-(trifluoromethyl)pyrazine-2-carboxylate had been formed in 32% yield.

### Example 5

### Oxidation with H₂O₂

To ethyl 4,4,4-trifluoro-2-(hydroxyimino)-3-oxobutanoate (0.176 g, approx 0.75 mmol, prepared according to Example 1) were added at 0° C. in the following order acetic acid (0.18 ml, 3.0 mmol), pyridine (1.2 ml, 15 mmol), ethylenediamine (0.065 ml, 0.98 mmol), and triethyl phosphite (0.193 ml, 1.13 mmol). The mixture was stirred at 0° C. for 1 h, and then at room temperature for 16 h. The mixture was then cooled to 0° C., hydrogen peroxide (50 wt-% in water, 0.17 ml, 3.0 mmol) was added, and the mixture was stirred at 0° C. for 1 h and at room temperature for 5.5 h. The mixture was then diluted with 1 N aqueous hydrochloric acid, saturated with sodium chloride, and extracted with ethyl acetate (3 ml). To the extract was added triisobutylphosphate (0.05 ml, 0.182 mmol) as internal standard. Analysis by 1H NMR indicated, that ethyl 3-(trifluoromethyl)pyrazine-2-carboxylate had been formed in 23% yield.

### Example 6

### One-pot, Oxidation with Br₂

To a mixture of acetic acid (4.8 ml, 80 mmol) and ethyl 4,4,4-trifluoro-3-oxobutanoate (2.92 ml, 20.0 mmol) at 5 to 15° C. was added dropwise a solution of sodium nitrite (1.79 g, 25.9 mmol) in water (2.32 ml) within 10 min. The mixture was stirred at 0° C. for 1 h, and nitrous gases were removed by bubbling nitrogen through the mixture for five minutes. Pyridine (30 ml, 0.37 mol) was added, and the mixture was cooled to 0° C. Ethylenediamine (1.736 ml, 26.0 mmol) was added dropwise, followed by the addition of triethyl phosphite (5.14 ml, 30.0 mmol). The mixture was stirred at 0° C. for 1 h, and then at room temperature for 15 h. The mixture was cooled to 0° C. and bromine (3.09 ml, 60.3 mmol) was added dropwise within 15 min. The mixture was stirred at 0° C. for 15 min, then at room temperature for 5 h. The mixture was diluted with brine (150 ml) and water (50 ml), and acidified by addition of aqueous concentrated hydrochloric acid (approximately 32 ml). The product was extracted with ethyl acetate (four times 50 ml), the combined extracts were dried over MgSO₄, filtered, and concentrated under reduced pressure to yield a mixture of triethyl phosphate and the title compound. Analysis by NMR indicated that the yield of ethyl 3-(trifluoromethyl)pyrazine-2-carboxylate was 46%.

### Example 7

### Ethyl 3-(trifluoromethyl)pyrazine-2-carboxylate with 2-methylpentanoic acid

To a stirred mixture of pyridine (1.62 ml, 20 mmol), 2-methylpentanoic acid (0.251 ml, 2.0 mmol), ethylenediamine (0.044 ml, 0.65 mmol), and triethyl phosphite (0.121 ml, 0.70 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 14 h. The mixture was then cooled to 0° C., and bromine (0.077 ml, 1.5 mmol) was added dropwise. After stirring at 0° C. for 30 min and then at room temperature for 2.5 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-trifluoromethyl)pyrazine-2-carboxylate was 51%.

### Example 8

### Ethyl 3-(trifluoromethyl)pyrazine-2-carboxylate with methanesulfonic acid

To a stirred mixture of pyridine (0.812 ml, 10 mmol), methanesulfonic acid (0.033 ml, 0.5 mmol), ethylenediamine (0.044 ml, 0.65 mmol), and triethyl phosphite (0.121 ml, 0.70 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 14 h. The mixture was then cooled to 0° C., and bromine (0.077 ml, 1.5 mmol) was added dropwise. After stirring at 0° C. for 30 min, and then at room temperature for 2.5 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 32%.

### Example 9

### Ethyl 3-(trifluoromethyl)pyrazine-2-carboxylate with N-hydroxysuccinimide

To a stirred mixture of pyridine (0.812 ml, 10 mmol), N-hydroxysuccinimide (231 mg, 2.0 mmol), ethylenediamine (0.044 ml, 0.65 mmol), and triethyl phosphite (0.121 ml, 0.70 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 14 h. The mixture was then cooled to 0° C., and bromine (0.077 ml, 1.5 mmol) was added dropwise. After stirring at 0° C. for 30 min, and then at room temperature for 2.5 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 25%.

### Example 10

### Ethyl 3-(trifluoromethyl)pyrazine-2-carboxylate with HCl

To a stirred mixture of pyridine (0.812 ml, 10 mmol), pyridine hydrochloride (116 mg, 1.0 mmol), ethylenediamine (0.044 ml, 0.65 mmol), and triethyl phosphite (0.121 ml, 0.70 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 22 h. The mixture was then cooled to 0° C., and bromine (0.077 ml, 1.5 mmol) was added dropwise. After stirring at 0° C. for 30 min, and then at room temperature for 5.5 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 15%.

### Example 11

### Ethyl 3-(trifluoromethyl)pyrazine-2-carboxylate with Benzoic Acid

To a stirred mixture of pyridine (1.62 ml, 20 mmol), benzoic acid (215 mg, 1.76 mmol), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 15.5 h. The mixture was then cooled to 0° C., and bromine (0.077 ml, 1.5 mmol) was added dropwise. After stirring at 0° C. for 30 min, and then at room temperature for 5.5 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 65%.

### Example 12

### Ethyl 3-(trifluoromethyl)pyrazine-2-carboxylate with 4-toluenesulfonic acid

To a stirred mixture of pyridine (1.62 ml, 20 mmol), 4-toluenesulfonic acid hydrate (95 mg, 0.5 mmol), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 15.5 h. The mixture was then cooled to 0° C., and bromine (0.077 ml, 1.5 mmol) was added dropwise. After stirring at 0° C. for 30 min, and then at room temperature for 5.5 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 43%.

### Example 13

### Ethyl 3-(trifluoromethyl)pyrazine-2-carboxylate with propionic acid

To a stirred mixture of pyridine (1.62 ml, 20 mmol), propionic acid (0.158 ml, 2.12 mmol), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 22 h. The mixture was then cooled to 0° C., and bromine (0.077 ml, 1.5 mmol) was added dropwise. After stirring at 0° C. for 1 h, and then at room temperature for 3 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 46%.

### Example 14

### Ethyl 3-(trifluoromethyl)pyrazine-2-carboxylate with dichloroacetic acid

To a stirred mixture of pyridine (1.62 ml, 20 mmol), dichloroacetic acid (0.145 ml, 1.76 mmol), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 22 h. The mixture was then cooled to 0° C., and bromine (0.077 ml, 1.5 mmol) was added dropwise. After stirring at 0° C. for 1 h, and then at room temperature for 3 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 39%.

### Example 15

### Ethyl 3-(trifluoromethyl)pyrazine-2-carboxylate with formic acid

To a stirred mixture of pyridine (1.62 ml, 20 mmol), formic acid (0.066 ml, 1.76 mmol), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 22 h. The mixture was then cooled to 0° C., and bromine (0.077 ml, 1.5 mmol) was added dropwise. After stirring at 0° C. for 1 h, and then at room temperature for 3 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 24%.

### Example 16

### Ethyl 3-(trifluoromethyl)pyrazine-2-carboxylate with benzoic acid and dichloromethane

To a stirred mixture of pyridine (0.812 ml, 10 mmol), benzoic acid (215 mg, 1.76 mmol), dichloromethane (0.812 ml), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 1 h, and then at room temperature for 22 h. The mixture was then cooled to 0° C., and bromine (0.077 ml, 1.5 mmol) was added dropwise. After stirring at 0° C. for 1 h, and then at room temperature for 3 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 51%.

### Example 17

### Ethyl 3-(trifluoromethyl)pyrazine-2-carboxylate with 2-chlorobenzoic acid

To a stirred mixture of pyridine (1.624 ml, 20 mmol), 2-chlorobenzoic acid (275 mg, 1.76 mmol), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 0.5 h, and then at room temperature for 14.5 h. The mixture was then cooled to 0° C., and bromine (0.077 ml, 1.5 mmol) was added dropwise. After stirring at 0° C. for 1 h, and then at room temperature for 2 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 55%.

### Example 18

### Ethyl 3-(trifluoromethyl)pyrazine-2-carboxylate with nicotinic acid

To a stirred mixture of pyridine (1.624 ml, 20 mmol), nicotinic acid (216 mg, 1.76 mmol), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 0.5 h, and then at room temperature for 14.5 h. The mixture was then cooled to 0° C., and bromine (0.077 ml, 1.5 mmol) was added dropwise. After stirring at 0° C. for 1 h, and then at room temperature for 2 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 20%.

### Example 19

### Ethyl 3-(trifluoromethyl)pyrazine-2-carboxylate with nicotinic acid N-oxide

To a stirred mixture of pyridine (1.624 ml, 20 mmol), nicotinic acid N-oxide (244 mg, 1.76 mmol), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 0.5 h, and then at room temperature for 14.5 h. The mixture was then cooled to 0° C., and bromine (0.077 ml, 1.5 mmol) was added dropwise. After stirring at 0° C. for 1 h, and then at room temperature for 2 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 21%.

### Example 20

### Ethyl 3-(trifluoromethyl)pyrazine-2-carboxylate with salicylic acid (2-hydroxybenzoic acid)

To a stirred mixture of pyridine (1.624 ml, 20 mmol), salicylic acid (243 mg, 1.76 mmol), ethylenediamine (0.044 ml, 0.65 mmol), and triethyl phosphite (0.129 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 0.5 h, and then at room temperature for 14.5 h. The mixture was then cooled to 0° C., and bromine (0.077 ml, 1.5 mmol) was added dropwise. After stirring at 0° C. for 1 h, and then at room temperature for 2 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 58%.

### Example 21

### Ethyl 3-(trifluoromethyl)pyrazine-2-carboxylate with pivalic acid (trimethylacetic acid) and trimethyl phosphite

To a stirred mixture of pyridine (3.24 ml, 40 mmol), pivalic acid (0.402 ml, 3.5 mmol), ethylenediamine (0.087 ml, 1.3 mmol), and trimethyl phosphite (0.177 ml, 1.5 mmol) at 0° C. was added ethyl 4,4,4-trifluoro-2-(hydroxyimino)-3-oxobutanoate (195 mg, 0.84 mmol, prepared according to Example 1). The mixture was stirred at 0° C. for 1 h, and then at room temperature for 22 h. The mixture was then cooled to 0° C., and bromine (0.154 ml, 3.0 mmol) was added dropwise. After stirring at 0° C. for 1 h, and then at room temperature for 2.5 h the mixture was diluted with 1N aqueous hydrochloric acid (12 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 48%.

### Example 22

### Ethyl 3-(trifluoromethyl)pyrazine-2-carboxylate by in situ generation of oxime with n-BuONO, benzoic acid, trimethyl phosphite (one pot)

To a mixture of ethyl 4,4,4-trifluoro-3-oxobutanoate (0.146 ml, 1.0 mmol) and n-butyl nitrite (0.129 ml, 1.1 mmol) at 0° C. was added acetyl chloride (0.079 ml, 1.1 mmol), and the mixture was stirred at 0° C. for 1 h and then at room temperature for 23 h. Benzoic acid (0.427 g, 3.5 mmol) was added, followed by the addition of pyridine (3.24 ml, 40 mmol) and ethylenediamine (0.087 ml, 1.3 mmol). The mixture was cooled to 0° C., and trimethyl phosphite (0.177 ml, 1.5 mmol) was added dropwise. The mixture was stirred at 0° C. for 1 h, and then at room temperature for 22 h. The mixture was then cooled to 0° C., and bromine (0.154 ml, 3.0 mmol) was added dropwise. After stirring at 0° C. for 1 h, and then at room temperature for 2.5 h the mixture was diluted with 1N aqueous hydrochloric acid (12 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 55%.

### Example 23

### Ethyl 3-(trifluoromethyl)pyrazine-2-carboxylate by generation of oxime with n-BuONO, benzoic acid, trimethyl phosphite, picoline

To a mixture of ethyl 4,4,4-trifluoro-3-oxobutanoate (1.46 ml, 10.0 mmol) and n-butyl nitrite (1.29 ml, 11.0 mmol) at 0° C. was added acetyl chloride (0.785 ml, 11.0 mmol), and the mixture was stirred at 0° C. for 6 h. More n-butyl nitrite (0.60 ml, 5.13 mmol) was added, and the mixture was stirred at room temperature for 15 h. Vacuum (40 mbar) was applied to remove volatiles. Analysis of the mixture (3.78 g) by 1H and 19F NMR indicated, that it mostly consisted of ethyl 4,4,4-trifluoro-2-(hydroxyimino)-3-oxobutanoate and butyl acetate. The density was 1.06 g/ml.

To a mixture of 3-methylpyridine (1.46 ml, 15.0 mmol), benzoic acid (213 mg, 1.75 mmol), ethylenediamine (0.043 ml, 0.65 mmol), and trimethyl phosphite (0.082 ml, 0.70 mmol) at 0° C. was added this mixture (0.197 ml containing 0.50 mmol oxime). The resulting mixture was stirred at 0° C. for 0.5 h and then at room temperature for 21 h. The mixture was then cooled to 0° C. and bromine (0.077 ml, 1.5 mmol) was added dropwise. After stirring at 0° C. for 1 h and at room temperature for 2 h the mixture was diluted with 1N HCl (10 ml), saturated with NaCl, and extracted with ethyl acetate (3 ml). Triisobutyl phosphate (0.060 ml, 0.219 mmol) was added as internal standard, and a concentrated sample was analyzed by 1H and 19F NMR. The yield of ethyl 3-(trifluoromethyl)pyrazine-2-carboxylate was 67%.

### Example 24

### Ethyl 3-(trifluoromethyl)pyrazine-2-carboxylate, oxidation with N,N′,N″-trichloroisocyanuric acid/Bu₄NI

To a stirred mixture of pyridine (1.20 ml, 15 mmol), benzoic acid (215 mg, 1.76 mmol), ethylenediamine (0.044 ml, 0.65 mmol), and trimethyl phosphite (0.089 ml, 0.75 mmol) at 0° C. was added dropwise a solution of ethyl 4,4,4-trifluoro-2-(hydroxyimino)-3-oxobutanoate (115 mg, 0.5 mmol, prepared according to Example 1), in pyridine (0.40 ml, 5.0 mmol). The mixture was stirred at 0° C. for 1 h, and then at room temperature for 16 h. The mixture was then cooled to 0° C., and tetrabutylammonium iodide (20 mg, 0.05 mmol) and N,N′,N″-trichloroisocyanuric acid (152 mg, 0.65 mmol) were added. After stirring at 0° C. for 0.5 h, and then at room temperature for 24 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 48%. o 25

### Ethyl 3-(trifluoromethyl)pyrazine-2-carboxylate, Oxidation with H₂O₂/Bu₄NI

To a stirred mixture of pyridine (1.20 ml, 15 mmol), benzoic acid (215 mg, 1.76 mmol), ethylenediamine (0.044 ml, 0.65 mmol), and trimethyl phosphite (0.089 ml, 0.75 mmol) at 0° C. was added dropwise a solution of ethyl 4,4,4-trifluoro-2-(hydroxyimino)-3-oxobutanoate (115 mg, 0.5 mmol, prepared according to Example 1), in pyridine (0.40 ml, 5.0 mmol). The mixture was stirred at 0° C. for 1 h, and then at room temperature for 16 h. The mixture was then cooled to 0° C., and tetrabutylammonium iodide (20 mg, 0.05 mmol) and hydrogen peroxide (50% in water, 0.114 ml, 2.0 mmol) were added. After stirring at 0° C. for 0.5 h, and then at room temperature for 24 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 28%. As byproduct, an N-oxide of ethyl 3-(trifluoromethyl)pyrazine-2-carboxylate was formed in 26% yield.

### Example 26

### Ethyl 3-(trifluoromethyl)pyrazine-2-carboxylate, oxidation with iodine; 30 eq pyridine, 3.0 eq benzoic acid

To a stirred mixture of pyridine (1.20 ml, 15 mmol), benzoic acid (184 mg, 1.51 mmol), ethylenediamine (0.044 ml, 0.65 mmol), and trimethyl phosphite (0.089 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 0.5 h, and then at room temperature for 15 h. The mixture was then cooled to 0° C., and iodine (386 mg, 1.5 mmol) was added. After stirring at 0° C. for 1 h, and then at room temperature for 5.5 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 66%. o 27

### Ethyl 3-(trifluoromethyl)pyrazine-2-carboxylate, Oxidation with H₂O₂/Na₂WO₄

To a stirred mixture of pyridine (1.60 ml, 19.8 mmol), benzoic acid (215 mg, 1.76 mmol), ethylenediamine (0.044 ml, 0.65 mmol), and trimethyl phosphite (0.089 ml, 0.75 mmol) at 0° C. was added ethyl 4,4,4-trifluoro-2-(hydroxyimino)-3-oxobutanoate (115 mg, 0.5 mmol, prepared according to Example 1). The mixture was stirred at 0° C. for 0.5 h, and then at room temperature for 15 h. The mixture was then cooled to 0° C., and sodium tungstate dihydrate (Na₂WO₄×2 H₂O; 15 mg, 0.045 mmol) and hydrogen peroxide (50% in water, 0.114 ml, 2.0 mmol) were added. After stirring at 0° C. for 1 h, and then at room temperature for 5.5 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3(trifluoromethyl)pyrazine-2-carboxylate was 28%. As byproduct, an N-oxide of ethyl 3-(trifluoromethyl)pyrazine-2-carboxylate was formed in 40% yield.

### Example 28

### Ethyl 3-(trifluoromethyl)pyrazine-2-carboxylate with hydroxybenzotriazole

To a stirred mixture of pyridine (1.62 ml, 20 mmol), 1-hydroxybenzotriazole hydrate (269 mg, 1.76 mmol), ethylenediamine (0.044 ml, 0.65 mmol), and trimethyl phosphite (0.089 ml, 0.75 mmol) at 0° C. was added ethyl 4,4,4-trifluoro-2-(hydroxyimino)-3-oxobutanoate (116 mg, 0.5 mmol, prepared according to Example 1). The mixture was stirred at 0° C. for 1.5, and then at room temperature for 20 h. The mixture was then cooled to 0° C., and bromine (0.077 ml, 1.5 mmol) was added dropwise. After stirring at 0° C. for 0.5 h, and then at room temperature for 2 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 49%.

### Example 29

### Ethyl 3-(trifluoromethyl)pyrazine-2-carboxylate with trifluoroacetic acid

To a stirred mixture of pyridine (1.62 ml, 20 mmol), trifluoroacetic acid (0.026 ml, 0.35 mmol), ethylenediamine (0.044 ml, 0.65 mmol), and trimethyl phosphite (0.089 ml, 0.75 mmol) at 0° C. was added ethyl 4,4,4-trifluoro-2-(hydroxyimino)-3-oxobutanoate (116 mg, 0.5 mmol, prepared according to Example 1). The mixture was stirred at 0° C. for 1.5, and then at room temperature for 20 h. The mixture was then cooled to 0° C., and bromine (0.077 ml, 1.5 mmol) was added dropwise. After stirring at 0° C. for 0.5 h, and then at room temperature for 2 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 39%.

### Example 30

### Ethyl 3-(trifluoromethyl)pyrazine-2-carboxylate with BF₃OEt₂

To a stirred mixture of pyridine (1.62 ml, 20 mmol), boron trifluoride diethyletherate (0.050 ml, 0.41 mmol), ethylenediamine (0.044 ml, 0.65 mmol), and trimethyl phosphite (0.089 ml, 0.75 mmol) at 0° C. was added ethyl 4,4,4-trifluoro-2-(hydroxyimino)-3-oxobutanoate (116 mg, 0.5 mmol, prepared according to Example 1). The mixture was stirred at 0° C. for 1.5, and then at room temperature for 20 h. The mixture was then cooled to 0° C., and bromine (0.077 ml, 1.5 mmol) was added dropwise. After stirring at 0° C. for 0.5 h, and then at room temperature for 2 h the mixture was diluted with 1N aqueous hydrochloric acid (10 ml), saturated with sodium chloride, and extracted with ethyl acetate (3 ml). An internal standard (triisobutyl phosphate) was added, and the extract was analyzed by NMR. The yield of ethyl 3-(trifluoromethyl)pyrazine-2-carboxylate was 29%.

### Example 31

### Dehydratization of Ethyl 4,4,4-trifluoro-2-(hydroxyimino)-3-oxobutanoate

The product of Example 1 (prepared according to Example 1, 2.07 g, approx 9.3 mmol) was mixed with dichloromethane (100 ml) and anhydrous calcium chloride (7.2 g, 64.9 mmol). The mixture was stirred at room temperature for 15 h.

Analysis of a sample by ¹H NMR in CDCl₃ indicated, that the dehydratization was complete. Filtration of the mixture over Celite and concentration under reduced pressure yielded 1.58 g of a turbid oil, which solidified completely after some days at room temperature. NMR confirmed that it was dehydrated ethyl 4,4,4-trifluoro-2-(hydroxyimino)-3-oxobutanoate. ¹H NMR (CDCl₃, 400 MHz) delta=4.43 (quart, J=7 Hz, 2H), 1.38 (t, J=7 Hz, 3H). ¹⁹F NMR (CDCl3, 376.5 MHz) delta=−72.8 (s).

### Example 32

### Ethyl 3-(trifluoromethyl)pyrazine-2-carboxylate by generation of oxime with n-BuONO, benzoic acid, trimethyl phosphite, picoline

To ethyl 4,4,4-trifluoro-3-oxobutanoate (1, 3.70 g, 20.1 mmol) at 0° C. were added n-butyl nitrite (2.81 ml, 24.0 mmol) and benzoic acid (366 mg, 3.00 mmol), and the mixture was stirred at 0° C. for 2 h and then at room temperature for 24 h. Vacuum (10 mbar, 40° C.) was applied for 0.5 h, to remove the excess butyl nitrite. The residue was the crude oxime. In a separate flask a mixture of benzoic acid (8.18 g, 67.0 mmol), 3-picoline (38 ml, 0.39 mol), and ethylenediamine (1.74 ml, 26.1 mmol) was prepared. Upon addition of the amine, a precipitate formed. After cooling to 0° C. trimethyl phosphite (3.31 ml, 28.0 mmol) was added, followed by the dropwise addition of the crude oxime. The oxime container was rinsed with 3-picoline (3.0 ml, 31 mmol), and this rinsing solution was also added to the reaction mixture. After stirring at 0° C. for 0.5 h and at room temperature for 3.5 h, the mixture was heated to 70° C. for 0.5 h and then cooled to 0° C. Bromine (2.56 ml, 50.0 mmol) was then added dropwise within 5 min. The mixture was stirred at 0° C. for 0.5 h, and then at room temperature for 1.5 h.

The mixture was cooled to 0° C. and added to an ice-cold mixture of concentrated aqueous hydrochloric acid (67 ml, 0.70 mol) and water (200 ml). After adding sodium bisulfite (3.12 g, 30.0 mmol), the product was extracted with butyl acetate (3 times with 50 ml), and the combined extracts were washed with brine (100 ml), with a solution of potassium carbonate (15 g, 0.11 mol) in water (100 ml), with brine (100 ml), were dried (MgSO₄) and concentrated under reduced pressure. The residue was purified by chromatography over silica gel (30 g; gradient elution with heptane/ethyl acetate from 100/0 to 8/2). Ethyl 3-(trifluoromethyl)pyrazine-2-carboxylate was obtained as an oil (1.81 g, 40% yield, 99% pure by ¹H NMR with triisobutyl phosphate as internal standard).
¹H NMR (CDCl₃, 400 MHz) delta 8.85 (m, 1H), 8.81 (m, 1H), 4.52 (q, J=7 Hz, 2H), 1.44 (t, J=7 Hz, 3H);
¹³C NMR (CDCl₃, 100 MHz) delta 163.7, 146.3, 145.3, 144.7, 141.6 (q, J=37 Hz), 120.6 (q, J=274 Hz), 63.2, 13.9;
¹⁹F NMR (CDCl₃, 377 MHz) delta −66.2.

### Example 33

### Ethyl 3-(trifluoromethyl)pyrazine-2-carboxylate WITH 2.2 equiv P(OMe)₃

To pyridine (1.2 ml, 15 mmol) was added benzoic acid (214 mg, 1.75 mmol) and ethylene diamine (0.040 ml, 0.60 mmol), and the mixture was cooled to 0° C. To this mixture were added ethyl 4,4,4-trifluoro-2-(hydroxyimino)-3-oxobutanoate (107 mg, 0.50 mmol) and then trimethyl phosphite (0.130 ml, 1.10 mmol). The resulting mixture was stirred at room temperature for 3.5 h, cooled to 0° C., and bromine (0.090 ml, 1.75 mmol) was added dropwise. After stirring at room temperature for 2 h the mixture was diluted with brine (15 ml), extracted (3 ml AcOEt), and triisobutyl phosphate (0.140 ml, 0.50 mmol) was added as internal standard to the organic phase. Analysis by ¹H NMR of a sample of the organic phase, after concentration by removal of parts of ethylacetate by evaporation, indicated, that ethyl 3-(trifluoromethyl)pyrazine-2-carboxylate had been formed in 71% yield.

### Example 34

### Ethyl 3-(trifluoromethyl)pyrazine-2-carboxylate WITH 1.8 equiv P(OMe)₃

To pyridine (1.2 ml, 15 mmol) was added benzoic acid (214 mg, 1.75 mmol) and ethylene diamine (0.040 ml, 0.60 mmol), and the mixture was cooled to 0° C. To this mixture were added ethyl 4,4,4-trifluoro-2-(hydroxyimino)-3-oxobutanoate (107 mg, 0.50 mmol) and then trimethyl phosphite (0.106 ml, 0.90 mmol). The resulting mixture was stirred at room temperature for 3.5 h, cooled to 0° C., and bromine (0.090 ml, 1.75 mmol) was added dropwise. After stirring at room temperature for 2 h the mixture was diluted with brine (15 ml), extracted (3 ml AcOEt), and triisobutyl phosphate (0.140 ml, 0.50 mmol) was added as internal standard to the organic phase. Analysis by 1H NMR of a sample of the organic phase, after concentration by removal of parts of ethylacetate by evaporation, indicated, that ethyl 3-(trifluoromethyl)pyrazine-2-carboxylate had been formed in 64% yield.

1. Method for the preparation of compound of formula (I); 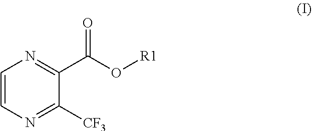
the method comprises two steps, a step ST1 and a step ST2;
ST1 comprises a reaction REAC1, wherein compound of formula (II) 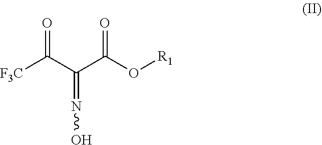
is reacted with ethylenediamine in the presence of an acid ACID1 and of a reducing agent REDUC1;
ST2 comprises a reaction REAC2, wherein the reaction product of REAC1 is reacted with an oxidant OXI2;
R1 is C₁₋₄ alkyl;
ACID1 is selected from the group consisting of BF₃OEt₂, hydroxybenzotriazole, nicotinic acid, nicotinic acid N-oxide, N-hydroxysuccinimide, formic acid, C₁₋₆ alkanoic acid, benzoic acid, benzoic acid substituted by Cl or OH, dichloroacetic acid, trifluoroacetic acid, trichloroacetic acid, toluenesulfonic acid, methanesulfonic acid, hydrochloric acid, sulfuric acid, and phosphoric acid;
REDUC1 is selected from the group consisting of zinc, iron, aluminum, magnesium, tin, SnCl₂, NaBH₄, hydrogen, PCl₃, H₃PO₂, P(R10)(R11)(R12) and P(OR20)(OR21)(OR22);
R10, R11, and R12 are identical or different and independently from each other selected from the group consisting of phenyl, tolyl, cyclohexyl, butyl, adamantyl, 2-carboxyethyl, and methyl;
R20, R21 and R22 are identical or different and independently from each other selected from the group consisting of H, C₁₋₈ alkyl, C₃₋₈ cycloalkyl, or phenyl;
OXI2 is selected from the group consisting of chlorine, bromine, iodine, N-chlorosuccinimide, N-bromosuccinimide, N-chlorophthalimide, N-bromophthalimide, N,N′,N″-trichloroisocyanuric acid, 1,3-dichloro-5,5-dimethylhydantoin, 1,3-dibromo-5,5-dimethylhydantoin, sodium hypochlorite, calcium hypochlorite, nitric acid, nitrous acid, sulfur, R30-OOH, air, and oxygen;
R30 is H or C₁₋₆ alkyl, the C₁₋₆ alkyl is unsubstituted or substituted with phenyl.

2. Method according to claim 1, wherein R1 is methyl or ethyl.

3. Method according to claim 1, wherein
ACID1 is selected from the group consisting of BF₃OEt₂, hydroxybenzotriazole, nicotinic acid, nicotinic acid N-oxide, N-hydroxysuccinimide, formic acid, acetic acid, propionic acid, n-butyric acid, iso-butyric acid, 2-methylpentanoic acid, pivalic acid, benzoic acid, chlorobenzoic acid, 2-hydroxybenzoic acid, dichloroacetic acid, trifluoroacetic acid, toluenesulfonic acid, methanesulfonic acid, and hydrochloric acid.

4. Method according to claim 1, wherein
REDUC1 is selected from the group consisting of zinc, aluminum, magnesium, hydrogen, PCl₃, H₃PO₂, P(R10)(R11)(R12) and P(OR20)(OR21)(OR22).

5. Method according to claim 1, wherein
REAC1 is done in a solvent SOLV1, SOLV1 is selected from the group consisting of acetonitrile, valeronitrile, dioxane, tert-butyl methyl ether, toluene, chlorobenzene, sulfolan, N,N-dimethylformamide, N,N-dimethylacetamide, 2-methyltetrahydrofuran, tetrahydrofuran, ethyl acetate, isopropyl acetate, butyl acetate, pyridine, 2-methylpyridine, 3-methylpyridine, 4-methylpyridine, 2-methyl-5-ethylpyridine, 2,4,6-trimethylpyridine, dichloromethane, chloroform, carbontetrachloride, and mixtures thereof.

6. Method according to claim 1, wherein
OXI2 is selected from the group consisting of chlorine, bromine, iodine, N-bromosuccinimide, N,N′,N″-trichloroisocyanuric acid, 1,3-dichloro-5,5-dimethylhydantoin, 1,3-dibromo-5,5-dimethylhydantoin, sodium hypochlorite, calcium hypochlorite, nitric acid, nitrous acid, R30-OOH, air, and oxygen.

7. Method according to claim 1, wherein
REAC2 is done in a solvent SOLV2, SOLV2 is selected from the group consisting of acetonitrile, valeronitrile, dioxane, tert-butyl methyl ether, toluene, chlorobenzene, sulfolan, N,N-dimethylformamide, N,N-dimethylacetamide, 2-methyltetrahydrofuran, tetrahydrofuran, ethyl acetate, isopropyl acetate, butyl acetate, pyridine, 2-methylpyridine, 3-methylpyridine, 4-methylpyridine, 2-methyl-5-ethylpyridine, 2,4,6-trimethylpyridine, dichloromethane, chloroform, carbontetrachloride, and mixtures thereof.

8. Method according to claim 1, wherein REAC1 and REAC2 are done consecutively in the same reaction vessel.

9. Method according to claim 1, wherein compound of formula (II) is prepared in a step ST0;
ST0 comprises a reaction REAC0, wherein compound of formula (III) 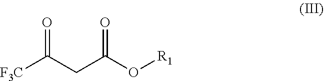
is reacted with NITR0 in the presence of ACID0;
NITR0 is selected from the group consisting of NaNO₂, ClNO, nitrosylsulfuric acid and R40-O—NO;
R40 is C₁₋₁₀ alkyl;
ACID0 is selected from the group consisting of ACID1 and chlorides and anhydrides of C₂₋₄ alkanoic acid.

10. Method according to claim 9, wherein
NITR0 is selected from the group consisting of NaNO₂, nitrosylsulfuric acid and R40-O—NO; and
R40 is C₁₋₅ alkyl.

11. Method according to claim 9, wherein
ACID0 is selected from the group consisting of ACID1 and chloride and anhydride of acetic acid.

12. Method according to claim 9, wherein
REAC0, REAC1 and REAC2 are done in the same reaction vessel.
